(19) United States
(12) Patent Application Publication
(10) Pub. No.: US 2009/0208573 A1
(43) Pub. Date: Aug. 20, 2009

(54) NOVEL POLYMORPH FORM OF IRBESARTAN

(75) Inventor: Ljubomir Antoncic, Ljubljana (SI)

Correspondence Address: ARENT FOX LLP, 1050 CONNECTICUT AVENUE, N.W., SUITE 400, WASHINGTON, DC, 20036, US

(73) Assignee: LEK PHARMACEUTICALS D.D, LJUBLJANA (SI)

(21) Appl. No.: 11/577,725

(22) PCT Filed: Nov. 9, 2005
(86) PCT No.: PCT/EP05/11982

§ 371 Date: Apr. 23, 2007

(30) Foreign Application Priority Data
Nov. 11, 2004 (SI) P200400308

(51) Int. Cl. A61K 9/28; C07D 257/04; A61K 31/41; A61K 31/5415; A61P 9/00

(52) U.S. Cl. 424/474; 548/250; 514/381; 514/223.5

(57) ABSTRACT

New form of irbesartan having favourable chargeability is prepared from the alcoholic/etheric or ketonic solution of irbesartan after slow cooling with sporadic or light agitation and alternatively new crystalline form of an acid addition salt of irbesartan is prepared from the aqueous solution of a sodium salt of irbesartan after strongly acidifying aforesaid solution and subsequently adjusting pH with an alkali. Those are used in manufacturing a pharmaceutical composition.

### FIELD OF THE INVENTION

The present invention relates to the novel forms of irbesartan and process for preparation thereof and to pharmaceutical compositions containing it.

### BACKGROUND OF THE INVENTION

Irbesartan is an antihypertensive agent known from EP 454511. From EP 708103, which discloses their X-ray spectra, two polymorphs are known where form A can be produced form a solvent system containing less than 10% of water, while Form B from a system with more than 10% of water. The specific morphological variant of form A can be prepared having properties as disclosed in EP 1089994. Additional form has been disclosed in WO 04089938. Amorphous irbesartan is known from WO 03050110. It is said that Irbesartan produced as taught in EP 454511 is a fluffy material with relatively low bulk and tap densities and undesirable flow characteristics, which consequently has unadvantageous electrostatic properties, among them a high chargeability as measured by tribugeneration between −30 and −40 nanocoulomb/g (10⁻⁹As/g). Alternatively irbesartan could be prepared by complex process using sonifications and/or temperature oscillations according to EP 1089994 to exhibit a chargeability as measured by tribugeneration between −0 and −10 nanocoulomb/g.

According to EP 454511 a solid composition in form of tablets is prepared by mixing the active ingredient with a vehicle such as gelatine, starch, lactose, magnesium stearate, talc, gum Arabic or the like and can be optionally-coated. The compositions containing from 20% to 70% by weight of irbesartan are known from EP 747050.

Irbesartan is administered in a unit dose up to 1 g, usually as the tablets containing from 75 mg up to 300 mg or 600 mg of the active ingredient. For the patient's compliance and comfort it is desirable to prepare tablets which are small and thus easy to swallow, however must have also suitable characteristics which are satisfactorily hardness, friability, disintegration needed for purpose of storage, handling and drug release characteristics.

To date, an irbesartan comprising pharmaceutical composition, such as tablet, which would incorporate a simply prepared active substance, and in which the proportion of the active substance would be as high as possible to give a relatively small easy swallowable tablet has not yet been realized.

### BRIEF DESCRIPTION OF THE DRAWINGS

FIGS. 1a and 1b are SEM photographs of two batches of the new form of irbesartan

FIG. 2 is an IR spectra of irbesartan hydrochloride.

FIG. 3 is an X-ray powder diffraction pattern of irbesartan hydrochloride.

FIG. 4 is a DSC thermogram of irbesartan hydrochloride.

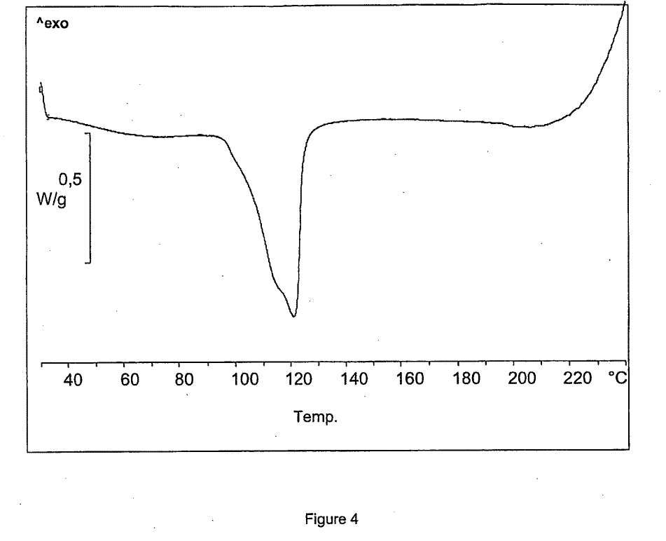

### DISCLOSURE OF THE INVENTION

The invention is in one aspect a form of irbesartan, which may be either 2-n-butyl-4-spirocyclopentane-1-[((2′-tetrazol-5-yl)biphenyl-4-yl)methyl]-2-imidazolin-5-one or its hydrochloride, characterized by the chargeability as measured by tribogeneration between −10 and −30, preferably between −15 and −27 nanocoulombs/g and/or by crystal habit such that the average ratio between the largest (length) and the smallest (width) dimension of the crystals is above 5, preferably above 10, more preferably above 11.

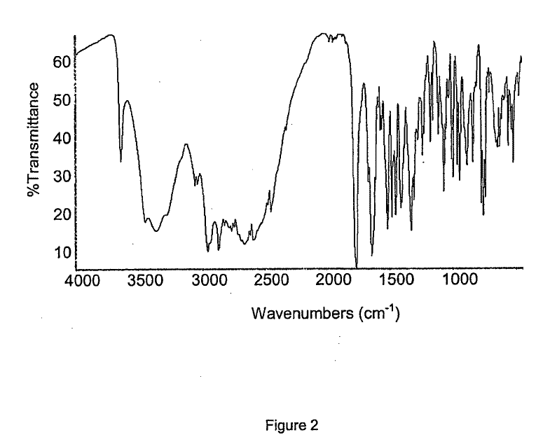

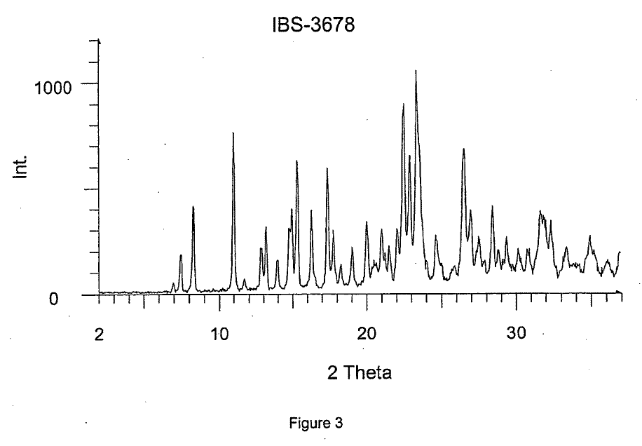

Second aspect of the invention is irbesartan hydrochloride having X-ray powder diffraction pattern comprising the most intense peaks at about 11.0; 15.3; 17.4; 22.5; 22.9; 23.4; 26.5°±0.2 2Theta, and preferably also one or more peaks at about 8.2; 12.8; 13.1; 14.9; 16.3; 17.8; 19.0; 20.0; 21.0; 21.5; 22.1; 24.7; 27.0; 27.5; 28.5; 29.4; 30.2; 31.7; 31.9; 32.3; 33.4; 34.9±0.2 2Theta and/or substantially as on FIG. 3 and/or IR spectra substantially as on FIG. 2.

Another aspect of the invention is a pharmaceutical composition comprising irbesartan wherein the amount of irbesartan in a composition is above 75% by weight, preferably below 85% by weight, which is manufactured using above described active pharmaceutical ingredient.

Specifically the composition may comprise a second active ingredient, preferably a hydrochlorotiazide and a pharmaceutically acceptable carrier, which may comprise one or more fillers or diluents, binders, disintegrants, glidants or antiadherents, lubricants and excipients that enhance the absorption of drugs.

In additional aspects the above binder is povidone; each diluent is selected form the group consisting of lactose monohydrate, microcrystalline cellulose and silicified microcrystalline cellulose; disintegrant is croscarmellose sodium; antiadherent is coloidal silicium dioxide; lubricant is sodium stearyl fumarate.

The aspect of the invention is a process for manufacturing the new form of irbesartan characterized by following steps:
a) providing a clear solution of irbesartan in a solvent selected from the group consisting of C1 to C4 aliphatic alcohol, THF and acetone, preferably ethanol or propanol, at the temperature above 45° C.;
b) subsequently cooling the solution to the temperature bellow 35° C. in a manner that at 40° C. the solution is clear; and
c) subsequently isolating the formed crystals of irbesartan.

Yet another aspect of the invention is a process for preparing the new crystalline from of irbesartan hydrochloride characterized by acidifying by HCl an aqueous solution of a sodium salt of irbesartan, which preferably consists of following steps:
a) acidifying aqueous solution of a sodium salt of irbesartan to pH below about 2;
b) adjusting the pH of above solution with sodium hydroxide to pH about 2; and
c) isolating the formed crystalline from of irbesartan hydrochloride.

Use of any the new crystalline form of irbesartan which is within a scope of the invention as a medicament is contemplated as an aspect therof, as well as method of treatment of hypertension by administering a therapeutically active amount thereof to the patients in need thereof.

In a specific aspect the above forms of irbesartan or irbesartan hydrochloride or any suitable form of irbesartan may be used for the manufacture of a medicament for treatment of hypertension; preferably in a process characterized by steps:
a) granulation with water a first granulate consisting of mixture of irbesartan, optionally a second active ingredient, one or more binders, a first portion of one or more diluents, and one or more disintegrants;
b) addition thereto an antiadherent, a second portion of one or more diluents and an lubricant and tableting; and
c) (optionally) coating the manufucatured tablets.

Preferably the amounts of above ingredients relative to the weight of the composition will be: irbesartan above 70%; the optional second active ingredient, which is preferably hydrochlorotiazide in amount from 3 to 6%; one or more diluents in first portion ifrom 2.5 to 7.5%, and in second portion from 1 to 8%; one or more binders from 4 to 12%; one or more disintegrants is from 2 to 6%; and one or more antiadherents and lubricants from 1 to 2%.

### DETAILED DESCRIPTION OF THE INVENTION

We have experimented with irbesartan form A formulating it by conventional methods by combining it with excipients such as gelatine, starch, lactose, magnesium stearate, talc, gum Arabic and realized that high loadings, that is the high proportion of the active substance versus total mass of the pharmaceutical composition are not possible.

Continuing our research we have surprisingly realized that active substance with relatively low bulk and/or tap densities and/or undesirable flow characteristics, (even or because of having a fluffy appearance), however exhibiting more favorable chargeability and/or crystal habit, can be formulated into pharmaceutical composition in high loadings above 50%, preferably above 70%, most preferably above 75%. The upper level of the loading depends on the excipients used and is believed to be up to 90%, preferably up to 85%. The new form of irbesartan, having appearance as seen by SEM of interconnected, interlaced or interwoven needles or lamellas or plates can be incorporated into a tablet even at higher loadings. The pharmaceutical composition may comprise above 70%, preferably above 75% and below 90% by weight of simply produced irbesartan which does not posses a high chargeability, where irbesartan has crystal habit such that the ratio between largest (length) and smallest dimension (width) of the crystals is above 5, preferably above 6, more preferably above 10, still more preferably above 11 and most preferably above 13.

Typical relatively low bulk densities are for example around 0.2, preferably 0.16 g/mL, while tap densities around 0.3, preferably 0.28 g/mL and undesirable flow characteristics are stickiness and adherence to surfaces, around in this context means ±0.05 g/mL.

Forms of irbesartan in accordance with our invention will comprise the compound 2-n-butyl-4-spirocyclopentane-1-[((2′-tetrazol-5-yl)biphenyl-4-yl)methyl]-2-imidazolin-5-one or its complexes, addition salts and other forms which do not affect the bioavailability, such as 2-n-butyl-4-spirocyclopentane-1-[((2′-tetrazol-5-yl)biphenyl-4-yl)methyl]-2-imidazolin-5-one hydrochloride or hydrobromide.

New forms of Irbesartan

A new form of irbesartan can be prepared having a crystal habit such that the ratio between the length and the width of the crystals is above 5:1, preferably above 10:1 and having favorable chargeability properties. In preferred embodiment more than 50%, preferably more than 65%, more preferably above 95% of all particles will exhibit described crystal habit. The percentage preferably relates to number of particles. Preferably it would exhibit same X-ray spectra as the substance prepared in EP 454511 and EP 1089994 (Form A as known from EP 708103). However the favorable chargeability properties means that substance in accordance with present invention does not posses a high electrostatic nature which would in one embodiment mean possessing a chargeability as measured by tribogeneration between −10 and −40 nanocoulomb/g, preferably between −10 and −30, nanocoulomb/g, more preferably between around 15 and 27 nanocoulombs/g .

One can prepare the new form of irbesartan from a hot clear solution of irbesartan. Hot solution will have temperature above 40° C., preferably above 45° C., more preferably around 60° C., most preferably the temperature of reflux of solvents. If the solution is so slowly cooled down so that the solution is still clear at around 40° C., the new form crystallizes. The process is surprising in that the crystals of new form will form even if the solution is agitated. The agitation can be for example sporadic shaking or stirring, for example with a suitable blade shaped stirrer at up to 40 rpm. After the solution has been cooled to 40° C. and it is still clear, the crystallization can be accelerated with seeding. The cooling can be continued below 40° C., preferably bellow 35° C., more preferably to room temperature (r. t.).

Preferably irbesartan should be completely dissolved in suitable solvent, preferably a C₁ to C₄ aliphatic alcohol, ether containing 2 to 6 C atoms, such as THF or C₃ to C₄ ketone, most preferably in ethanol, i-propanol, THF or acetone. As log as the complete dissolution is achieved, the concentration may vary for example from 1 g in 5 mL to 100 mL, preferably in alcohols from 1 g in 10 to 25 mL. The essential part of the invention is a slow rate of cooling of the clear solution, in one embodiment not exceeding 20° C., preferably 10° C. per hour, more preferably even slower and in other embodiment taking from the temperature of reflux to the r.t. up to 10 hours, preferably 2 to 3 hours.

The yield and/or favorable physical properties (those allowing easy incorporation into a pharmaceutical composition) can be further improved in another embodiment of the invention, where small amount of the crystal prepared in a separate experiment as above are used as a seed for crystallization of irbesartan from a solution.

Alternatively irbesartan having desired properties, i.e. crystalline substance having the ratio of longest and shortest crystal dimension above 5:1, preferably above 10:1, and/or favorable chargeability may be prepared as an acid addition salt. In this manner for example irbesartan hydrochloride can be obtained from an aqueous solution of a sodium salt of irbesartan by acidifying said aqueous solution by HCl. Not committing to the theory it is believed that in this manner the chargeability is influenced by electron density of relatively large anion, as compared to intrinsic effects caused by the inter-correlation of slowly crystallized particles.

Thus in this alternative sodium salt should substantially be dissolved and its concentration may for example lay within a range of 0.05 g/mL to 0.5 g/mL The starting solution may be prepared by conventional means. For example by removal a protecting group, such as trityl from trityl irbesartan by an alkali containing sodium ions. After the addition of hydrochloric acid, preferably in an excess, and additional adjustment of pH to about 1 to 4, preferably to about pH around 2 and partial evaporation of a solvent, preferably careful evaporation above room temperature, most preferably vacuum evaporation at about 50° C. one obtains crystalline irbesartan hydrochloride, which is for example characterized by an X-ray powder diffraction pattern substantially as depicted on FIG. 3 or having the peaks at 2 to 5, preferably 10 or more characteristic values that are selected from values at about 8.2; 11.0; 12.8; 13.1; 14.9; 15.3; 16.3; 17.4; 17.8; 19.0; 20.0; 21.0; 21.5; 22.1; 22.5; 22.9; 23.4; 24.7; 26.5; 27.0; 27.5; 28.5; 29.4; 30.2; 31.7; 31.9; 32.3; 33.4; 34.9±0.2° 2Theta. Of those are the most intense peaks at about 11.0; 15.3; 17.4; 22.5; 22.9; 23.4; 26.5°±0.2 2Theta. It is further characterized by DSC, IR spectra and m.p.: 110-123° C. Morphologically it is a free-flowing powder in form of lamellas or plates which do not exhibit same physical properties as the substance produced according to the prior art, for example the flowing properties indicate favourable chargeability.

The new forms of irbesartan can be produced in substantially pure form or in a mixture with any other form. Depending on the desired characteristic, for example desired dissolution properties, the substantially pure polymorph forms can be incorporated into a pharmaceutical composition in pure form or alternatively a mixture thereof.

The described new forms of irbesartan, alone or in combination with another active, e.g. diuretic, preferably hydrochlorotiazide, have a potent antihypertensive activity and incorporated into a pharmaceutical composition can be in a form suitable for peroral or parental application. Pharmaceutical composition in accordance with this invention can be embodied for example in form of tablet, capsules, pellets, granules and supozitories or their combined forms. Solid pharmaceutical compositions (dosage forms) can be shielded, for example coated with the aim of increasing peletibility or regulating the disintegration or absorption.

The solid dosage forms comprising new forms of irbesartan can be prepared by conventional method. The favorable electrostatic properties and/or crystal habit allow the formation of a tablet which is convenient to the patient. Since irbesartan is administered in dosage up to 600 mg per tablet, this means that only small amount of excipients (constituting a pharmaceutically acceptable carrier) may be used. Tablet can be for example manufactured by direct compression though wet granulation is another commonly used technique. In wet granulation at least one of the ingredients can be mixed or contacted with liquid and further processed to provide aggregates, the liquid can be partially or completely removed and optionally other or more of the same ingredients may be further added and solid dosage forms manufactured.

Tableting compositions may have in addition to active pharmaceutical ingredient few or many components, depending upon the tableting method used, the release rate desired and other factors. For example, compositions of the present invention may contain inactive ingredients (excipients) which function as such as different fillers, binders, disintegrants, glidants, lubricants, antiadherents and excipients that enhance the absorption of drugs from gastrointestinal tract.

Suitable fillers or diluents may be selected from microcrystalline cellulose, powdered cellulose, lactose, starch, pregelatinized starch, sucrose, glucose, mannitol, sorbitol, calcium phosphate, calcium hydrogen phosphate, aluminium silicate, sodium chloride, potassium chloride, calcium carbonate, calcium sulphate, dextrates, dextrin, maltodextrin, glycerol palmitostearate, hydrogenated vegetable oil, kaolin, magenesium carbonate, magnesium oxide, polymethacrylates, talc, and others. Preferred fillers are microcrystalline cellulose and lactose, most preferable (optionally silicified) microcrystalline cellulose and lactose monohydrate. Suitable binders may be starch, pregelatinized starch, gelatine, sodium carboxymethylcellulose, polyvinylpyrrolidone, alginic acid, sodium alginate, acacia, carbomer, dextrin, ehylcellulose, guar gum, hydrogenated vegetable oil, methylcellulose, hydroxyethylcellulose, hydroxypropylcellulose, hydroxypropylmethylcellulose, glucose syrup, magnesium aluminium silicate, maltodextrin, polymethacrylates, zein. Preferably hydroxypropyl cellulose, hydroxypropyl methylcellulose and polyvinylpyrrolidone, most preferably polyvinylpyrrolidone are used. Suitable disintegrants may be selected from starch, pregelatinized starch, sodium starch glycolate, sodium carboxymethylcellulose, cross-linked sodium carboxymrethylcellulose, calcium carboxymethylcellulose, methylcellulose, microcrystalline cellulose, powdered cellulose, polacrilin potassium, cross-linked polivinylpyrrolidone, alginic acid, sodium alginate, colloidal silicon dioxide, guar gum, magnesium aluminium silicate, and others. Preferred disintegrants are sodium starch glycolate, cross-linked carboxymethylcellulose sodium, croscarmellose sodium and cross-linked polyvinylpyrrolidone, most preferably croscarmellose sodium. Suitable glidants may be magnesium stearate, calcium stearate, aluminium stearate, stearic acid, palmitic acid, cetanol, stearol, polyethylene glycols of different molecular weights, magnesium trisilicate, calcium phosphate, colloidal silicon dioxide, talc, powdered cellulose, starch and others. Preferred antiadherent is colloidal silicilon dioxide. Suitable lubricants may be selected from stearic acid, calcium, magnesium, zinc or aluminium stearate, siliconized talc, glycerol monostearate, glycerol palmitostearate, hydrogenated castor oil, hydrogenated vegetable oil, mineral oil, light mineral oil, polyethylene glycol, sodium benzoate, sodium lauryl sulphate, sodium stearyl fumarate, talc and others. Preferred lubricants are calcium or magnesium stearate and stearic acid, most preferable is sodium stearyl fumarate. Suitable absorption enhancers may be selected from surface active agents, fatty acids, middle chain glycerides, steroide detergents (salts of bile salts), acyl carnitine and alcanoloil choline (esters of carnitine and choline and fatty acids with middle chain and long chain), N-acyl derivatrives of alpha-amino acids and N-acyl derivatives of non-alpha-amino acids, chitosanes and other mucoadhesive polymers. Especially suitable absorption enhancers are sodium deoxycholate, sodium taurocholate, polisorbate 80, sodium lauryl sulfate, sodium dodecylsulfate, octanoic acid, sodium docusate, sodium laurate, glyceride monolaurate, stearic acid, palmitinic acid, palmitooleinic acid, glycerilmonooleate, sodium taurocholate, ethylenediaminetetraacetic acid, sodium edentate, sodium citrate, b-cyclodextrine and sodium salicylate.

In accordance with our invention one can mix irbesartan with relatively small amount of excipients selected as described bellow, preferably so that ratio of irbesartan to excipients is above 5 to 1, preferably above 6 to 1 and subsequently granulate the mixture with suitable granulating liquid. Any granulating liquid can be selected based on solubilities of irbesartan and excipinets, however water a preferred. Surprisingly the granulate after drying does not any more exhibit undesirable flow characteristics and can be optionally with addition of a small amount of any additional excipients formulated into tablets, onto which a coating can be applied. For the film coat standard excipients are used that are most suitable for water film-coating formulation such as hypromellose and hydroxypropylcellulose as film formers, polethyleneglycol as plasticizer, lactose as soluble filler, titanium dioxide as white coloring agent and talc as anti-sticking agent. Other coloring agent such as ferric oxide can be added.

In the preferred embodiment the tablets containing 300 mg of irbesartan will not weight more than 400 mg, and can be prepared by granulating with water first granulate consisting of mixture of irbesartan, optionally a second active ingredient, such as hydrochlorotiazide in amount about 3 to 6%; a binder, preferably in amount from 2.5 to 7.5%, more preferably about 5%; one or more diluents, preferably in amount from 4 to 12%, more preferably about 8%, a disintegrant, preferably in amount from 2 to 6%, more preferably about 4%. To said first granulate extragranular excipients: antiadherent in amount about 1%; one or more diluents in amount from about 1 to 8% diluent and an lubricant in amount of about 2% are added and mixture tableted. Analogously a tablet containing 150 mg of irbesartan will not weight more than 200 mg. All amounts are weight percents relative to the mass of the tablet and the term about means an amount ±20% , preferably ±10% relative to the amount. Preferably the coating will not be more than about 1 to 2% by weight to the composition.

### EXPERIMENTAL PART

Infrared spectra was obtained with Nicolet Nexus FTIR spectrophotometer. Samples were analyzed in KBr and scanned from 400 to 4000 cm⁻¹ with 16 scans and 2 cm⁻¹ resolution. Thermogram was obtained with Mettler Toledo DSC822^{e} differential scanning calorimeter. The sample was placed in an unsealed aluminium pan with one hole and heated at 5° C./min in the temperature range from 30° C. to 240° C. in the nitrogen (100 mL/min).

Powder X-ray diffraction spectra of the sample was recorded on Philips PW1710 with reflexion technique: CuKa radiation, range from 2° to 37° 2Theta, step 0.04° 2Theta, integration time 1 sec.

From an X-ray diffraction pattern of a powdery substance one can establish differences among different crystal lattices, and can obtain information on level of order i.e. crystallinity where lover crystallinity causes peaks to broaden. The diffraction values for a crystalline substance will be substantially independent of the difractometer used, if the difractometer is calibrated the values can differ for about 0.05° 2 Theta, taking into account the rounding the differences in values lay in the order of ±0.1° 2Theta, however the different recording conditions or differences in preparing or handling samples can cause the variations from the values reported for as much as ±0.2 2Theta. The intensities of each specific diffraction peak are a function of various factors, one of those being a particle size and preferred orientation. Skilled person will recognize the polymorph form from the comparison od whole X-ray powder diffraction patterns and specifically from the strongest peaks or any two to five or more distinct peaks selected from the listed peaks.

A method of measuring electrostatic nature is a tribogeneration where relative charge on the powdered sample is measured with an electrometer equipped with a Faraday cup (NanoCoulumb Meter, Model 284, Monroe Electronics, USA). Prior to measurement, the charge is generated by strongly vibrating the sample (approximately 3 g) on a dry Petri dish (D=24 cm) for 2 minutes. The charged sample is then transferred to a Faraday cup of the electrometer with a plastic spoon and its charge is measured by an electrometer. After the charge measurement the sample is weighted and the relative charge calculated. All measurements are performed at controlled dry atmosphere conditions (15% relative humidity and 23° C.). The substances in accordance with present invention preferably exhibits values between around −27 in certain embodiments and around −15 nanocoulomb/g in other embodiments, compared to values −30 to −40 nanocoulomb/g and 0 to −10 nanocoulomb/g for previously known forms

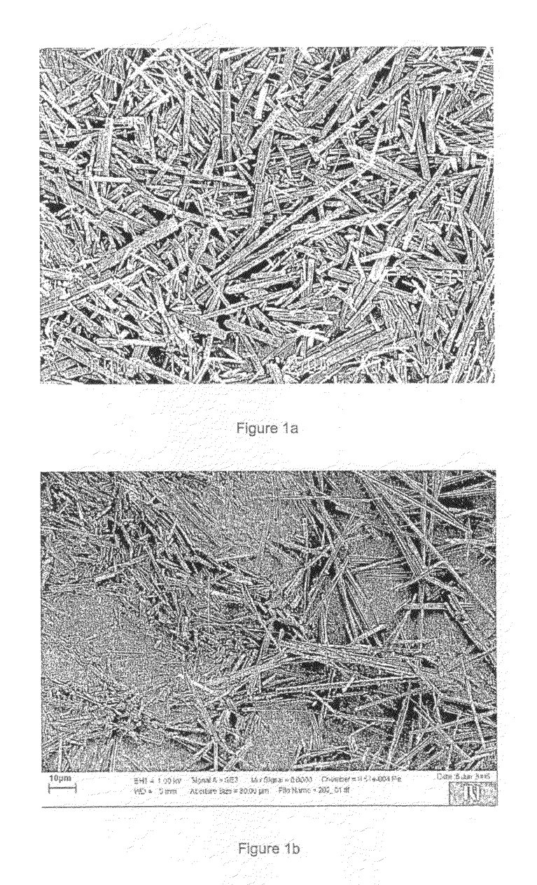

The chargeability so measured is −26.5±6·10⁻⁹ As/g for the sample of the irbesartan in accordance with our invention corresponding to FIG. 1a (1649) and 45.8±4.9·10⁻⁹As/g in FIG. 1b (3913D) with magnification 500×.

The shape of crystals is determined by an analysis of an image, taken by the scanning electron microscope (SEM), JEOL JXA840A at magnification 500-2000×. The length/width ratio of the crystal habit is determined by measuring length and width of a representative sample (N=200) of crystals on the image, using the image analyzing software Olympus DP-Soft, v-3.2. The length/width ratio is calculated independently for each crystal. Finally, the number average of the ratio is determined and optionally histogram calculated.

The following table shows the properties of different batches of irbesartan:

| | Solvent from which | | | | a:b | |
|---|---|---|---|---|---|---|
| | the substance is | | | | conf. | chargeab. |
| | crystalized | a (μm) | b (μm) | a:b | int. 95% | 10⁻⁹ As/g |
| 1 | n-propanol | 14.9 ± 10.1 | 1.2 ± 1.4 | 13 ± 10 | 11.25-15.15 | −33.7 ± 15 |
| 2 | i-propanol | 78 ± 68 | 6.7 ± 2.8 | 13.4 ± 1.9 | 11.1-15.7 | −32 ± 4.6 |
| 3 | acetone | 20.7 ± 17.4 | 1.0 ± 0.5 | 23.3 ± 23 | 18.6-27.8 | −45.8 ± 4.9 |
| 4 | methanol | 130 ± 96 | 7.9 ± 3.4 | 17 ± 12.9 | 14.6-20.4 | −27.5 ± 5.6 |
| 5 | abs. ethanol | 58 ± 50 | 6.4 ± 1.2 | 9.5 ± 8 | 8.1-11.1 | −17.1 ± 2.9 |
| 6 | abs. ethanol * | 32 ± 32 | 1.4 ± 0.6 | 22 ± 18 | 15.8-27.4 | −27.9 ± 2.8 |
| 7 | 96% ethanol * | 21 ± 17 | 2.6 ± 1.0 | 13 ± 9 | 10.1-15.9 | −26.5 ± 6 |

* experiment with seeding
Other batches, especially crystallized from THF and acetone had a:b ratios for example 4 ± 3; 6.1 ± 3.7 and chargeability −14.2 ± 2; −15.6 ± 2.3 · 10⁻⁹ As/g respectively.

In the preferred embodiment our invention is a process where 15 g of irbesartan is completely dissolved in 300 mL of absolute ethanol heated to temperature of reflux to provide a clear solution which is subsequently cooled during 2 to 3 hours to reach room temperature. While the solution is cooled, it is stirred all the time at 40 rpm. The rate of cooling and agitation is preferably such that while passing the temperature of 40° C. the solution is still clear. Bellow the temperature of 40° C. the crystals will begin to form and the rate of crystallization can optionally be accelerated by seeding. The formed crystals are separated by filtration or centrifugation and dried, optionally under vacuum and optionally at the elevated temperature, such as 55° C.

In another preferred embodiment irbesartan hydrochloride is prepared by providing a clear solution of about 1 g of sodium salt of irbesartan in about 10 mL of water into which an excess of hydrochloric acid, preferably 7.7 mL or more of 1 N HCl and adjustment of pH to about 1 to 4, preferably to about pH around 2 and partial evaporation of a solvent to yield saturated solution and cooling one obtains crystalline irbesartan hydrochloride

Following examples further illustrate the invention, They are provided for illustrative purposes only and are not intended to limit in any way the invention.

### EXAMPLE 1

A stirred suspension of irbesartan (5 g) in n-propanol (100 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at temperature of 60° C. to obtain 3.6 g of product.

### EXAMPLE 2

A stirred suspension of irbesartan (5 g) in i-propanol (200 mL) was heated at the temperature of reflux until clear solution was obtained. Solution was filtered and slowly cooled with periodically shaking (or slow stirring at 40 rpm) to a temperature of about 20° C. in a period of 2-3 hours. Crystalline suspension was filtered and crystals were dried in vacuo at a temperature of 60° C. to obtain 4.16 g of product.

### EXAMPLE 3

A stirred suspension of irbesartan (5 g) in acetone (500 mL) was heated at the temperature of reflux until clear solution was obtained. Solution was filtered and slowly cooled with periodically shaking (or slow stirring at 40 rpm) to a temperature of about 20° C. in a period of 2-3 hours. Crystalline suspension was filtered and crystals were dried in vacuo at a temperature of 60° C. to obtain 2.16 g of product.

### EXAMPLE 4

A stirred suspension of irbesartan (5 g) in metanol (50 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at a temperature of 60° C. to obtain 4.2 g of product.

### EXAMPLE 5

A stirred suspension of irbesartan (5 g) in absolute ethanol (100 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at a temperature of 60° C. to obtain 4.11 g of product.

### EXAMPLE 6

A stirred suspension of irbesartan (15 g) in absolute ethanol (300 mL) was heated at the temperature of reflux until clear solution was obtained. Solution was slowly cooled with periodically shaking (or slow stirring at 40 rpm) to a temperature of about 20° C. in a period of 2-3 hours. Meanwhile still clear solution was seeded at a temperature of about 40° C. Crystalline suspension was filtered and crystals were dried in vacuo at a temperature of 55° C. to obtain 12.28 g of product.

### EXAMPLE 7

A stirred suspension of irbesartan (15 g) in 96% ethanol (300 mL) was heated at the temperature of reflux until clear solution was obtained. Solution was slowly cooled with periodically shaking (or slow stirring at 40 rpm) to a temperature of about 20° C. in a period of 2-3 hours. Meanwhile still clear solution was seeded at a temperature of about 50° C. Crystalline suspension was filtered and crystals were dried in vacuo at a temperature of 55° C. to obtain 11.94 g of product.

### EXAMPLE 8

A stirred suspension of irbesartan (15 g) in n-propanol (174 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at a temperature of 55° C. to obtain 12.18 g of product.

### EXAMPLE 9

A stirred suspension of irbesartan (15 g) in i-propanol (330 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at a temperature of 55° C. to obtain 13.50 g of product.

### EXAMPLE 10

A stirred suspension of Irbesartan (15 g) in acetone (1050 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at a temperature of 55° C. to obtain 10.20 g of product.

### EXAMPLE 11

A stirred suspension of irbesartan (15 g) in tetrahydrofuran (255 mL) was heated at the temperature of reflux until clear solution was obtained. Solution was slowly cooled with periodically shaking (or slow stirring at 40 rpm) to a temperature of about 20° C. in a period of 2-3 hours. Crystalline suspension was filtered and crystals were dried in vacuo at a temperature of 55° C. to obtain 11.4 g of product.

### EXAMPLE 12

23.25 g of tritylirbesartan was dissolved in 90 mL of THF. To a clear solution 90 mL of methanol, 1 mL of water and 0.6 g p-toluenesulphonic acid was added. Reaction mixture was stirred at the temperature of reflux for 24 hours. Solvent was evaporated and the volume was replenished by addition of water, and ph was adjusted to ph12.7 with NaOH (30% aq). Reaction mixture was extracted with 150 mL of diethylether, 150 mL of toluene and 150 mL of diethyl ether successively. Layers were separated. Water layer was acidified with. approximately 116 mL of1 N HCl in one portion; mixture was stirred at room temperature until white suspension was obtained. pH of suspension was adjusted to pH 2 with 30% NaOH and filtered. Product was washed with 30 mL of ethyl acetate and vacuum dried at 50° C. Yield: 14 g of a substance in new crystalline form.

### EXAMPLE 13

3 g of irbesartan was suspended in 36 mL of water, pH was adjusted to ph 12.69 with cca 1 mL of 40% NaOH at room trmperature. Clear solution was obtained. To this solution 25 mL of 1 M HCl was added in one portion at room temperature with stirring. Mixture was stirred at room temperature until white suspension was obtained. pH of suspension was adjusted to ph 2.00 with 30% NaOH and filtered. Product was washed with 10 mL of water and vacuum dried at 50° C. Yield 3.13 g of a substance in new crystalline form with melting point (Kofler microstage)110° C.-123° C.

### FORMULATION EXAMPLE 1

A tablet is prepared as follows: First granulate is prepared by mixing: irbesartan 300.00 mg and polyvinylpyrrolidone (K 25) 19.70 mg and lactose (70-100 mesh) 18.74 mg and microcrystalline cellulose (Avicel PH 101) 9.85 mg together with Na carboxy methyl cellulose (AcdiSol) 13.79 mg and granulating with water. Thereto silicon dioxide (Aerosil 200) 2.36 mg, sSilicified microcrystalline cellulose (Prosolv HD90) 21.67 mg and Na stearil fumarate (Pruv) 7.88 mg are added and tableted.

### FORMULATION EXAMPLE 2

A tablet is prepared by making first granulate as above which contains additionally 2.50 mg hydrochlorotiazide Thereto silicon dioxide (Aerosil 200) 2.36 mg, silicified microcrystalline cellulose (Prosolv HD90) 9.17 mg and Na stearil fumarate (Pruv) 7.88 mg are added and tableted.

### FORMULATION EXAMPLE 3

A tablet is prepared by granulating with water first granulate consisting of irbesartan 150.00 mg, hydrochlorotiazide 12.50 mg and polyvinylpyrrolidone (K 25) 9.85 mg and lactose (70-100 mesh) 3.12 mg and microcrystalline cellulose (Avicel PH 101) 4.92 mg together with Na carboxy methyl cellulose (AcdiSol) 6.90 mg. Thereto silicon dioxide (Aerosil 200) 1.18 mg, silicified microcrystalline cellulose (Prosolv HD90) 4.58 mg and Na stearil fumarate (Pruv) 3.94 mg are added and tableted.

Although the present formulations contains high percentage of active substance alone or in combination with another active, rapid and complete drug release is achieved.

Accelerated stability studies show that the formulation exhibits good stability in the packaging that protects product from moisture.

1. A process for preparing irbesartan which is 2-n-butyl-4-spirocyclopentane-1-[((2′-tetrazol-5-yl)biphenyl-4-yl)methyl]-2-imidazolin-5-one comprising:
a) providing a clear solution of irbesartan in a solvent selected from the group consisting of C1 to C4 aliphatic alcohol, THF and acetone at the temperature above 45° C.;
b) cooling the solution to the temperature bellow 35° C. in a manner that at 40° C. the solution is clear;
c) isolating the formed crystals of irbesartan.

2. The process according to claim 1, wherein step a) irbesartan is completely dissolved in said solvent.

3. The process according to claim 1 wherein the solution of step b) is slowly cooled at rate not exceeding 10° C. per hour.

4. The process according to claim 1 wherein the solution of step a) is seeded.

5. The process according to claim 1 wherein the concentration of a clear solution is from 1 g of irbesartan in 5 mL to 100 mL of solvent.

6. The process according to claim 5 wherein the concentration of a clear solution is from 1 g of irbesartan in 10 mL to 25 mL of solvent, wherein the solvent is selected from the group consisting of ethanol, propanol and mixture thereof.

7. The process according to claim 1 wherein the solution is during cooling in step b) agitated by stirring where the stirring does not exceed 40 rpm.

8. The process according to claim 1 wherein the isolated crystals of irbesartan exhibit X-ray spectra of Form A.

9. The process according to claim 1 wherein the isolated crystals of irbesartan have chargeability as measured by tribogeneration between −15 and −27 nanocoulombs/g.

10. The process according to claim 1 wherein the isolated crystals of irbesartan have crystal habit such that the average ratio between the largest and the smallest dimension of the crystals is above 10:1.

11. The process according to claim 10 wherein the isolated crystals of irbesartan have crystal habit such that the average ratio between the largest and the smallest dimension of the crystals is above 11:1.

12. The process according to claim 1 wherein the isolated crystals of irbesartan have appearance as seen by SEM of interconnected, interlaced or interwoven needles.

13. The process according to claim 1 wherein the isolated crystals of irbesartan have appearance as seen by SEM as on FIG. 1.

14. The process according claim 1 wherein the isolated crystals of irbesartan have bulk density around 0.2 g/mL.

15. The process according to claim 1 wherein the isolated crystals of irbesartan have tap density around 0.3 g/mL.

16. A crystalline form of irbesartan, which is 2-n-butyl4-spirocyclopentane-1-[((2′-tetrazol-5-yl)biphenyl-4-yl)methyl]-2-imidazolin-5-one, and exhibits the same X-ray spectra as Form A of irbesartan, characterized in that the chargeability as measured by tribogeneration is between −10 and −30 nanocoulombs/g.

17. The crystalline form of irbesartan according to claim 16 wherein the chargeability as measured by tribogeneration is between −15 and −27 nanocoulombs/g.

18. The crystalline form of irbesartan according to claim 16 characterized by crystal habit such that the average ratio between the largest and the smallest dimension of the crystals is above 5:1.

19. The crystalline form of irbesartan according to claim 18 characterized by crystal habit such that the average ratio between the largest and the smallest dimension of the crystals is above 10:1.

20. The crystalline form of irbesartan according to claim 19 characterized by crystal habit such that the average ratio between the largest and the smallest dimension of the crystals is above 11:1.

21. (canceled)

22. (canceled)

23. (canceled)

24. (canceled)

25. (canceled)

26. A pharmaceutical composition comprising a crystalline form irbesartan according to claim 16 in amount above 70% relative to the weight of the composition, and optionally from 3 to 6% relative to the weight of the composition of a second active ingredient, and from 3.5 to 15.5% relative to the weight of the composition of one or more binders, and from 4 to 12% relative to the weight of the composition of one or more diluents, from 2 to 6% relative to the weight of the composition of one or more disintegrants, and from 1 to 2% relative to the weight of the composition of one or more antiadherents and lubricants.

27. A pharmaceutical composition according to previous claim, characterized in that it is manufactured in a process consisting of steps:
a) granulating with water a first granulate consisting of mixture of crystalline form of irbesartan, according to claim 16, optionally a second active ingredient, one or more binders, a first portion of one or more diluents, and one or more disintegrants;
b) adding an antiadherent, a second portion of one or more diluents and an lubricant and tableting; and
c) (optionally) coating the tablets.

28. A pharmaceutical composition according to claim 27, characterized in that the binder is povidone.

29. A pharmaceutical composition according to claim 27, wherein each of the diluents is selected from the group consisting of lactose monohydrate, microcrystalline cellulose and silicified microcrystalline cellulose.

30. A pharmaceutical composition according to claim 27, the disintegrant is croscarmellose sodium.

31. A pharmaceutical composition according to claim 27, wherein the antiadherent is coloidal silicium dioxide.

32. A pharmaceutical composition according to claim 27, wherein the lubricant is sodium stearyl fumarate.

33. A pharmaceutical composition according to claim 27, wherein the second active ingredient is hydrochlorotiazide.

34. A process for manufacturing a pharmaceutical composition comprising irbesartan according to claim 16, wherein said process comprises:
a) granulating with water a first granulate consisting of mixture of irbesartan, optionally a second active ingredient, one or more binders, a first portion of one or more diluents, and one or more disintegrants;
b) adding thereto an antiadherent, a second portion of one or more diluents and an lubricant and tableting; and
c) (optionally) coating the tablets.

35. A process according to claim 34 wherein the amount of irbesartan is above 75% relative to the weight of the composition, and the second active ingredient is hydrochlorotiazide which is optionally present in amount from 3 to 6% relative to the weight of the composition, and the amount of one or more diluents in first portion is from 2.5 to 7.5% relative to the weight of the composition, and the amount of one or more diluents in second portion is from 1 to 8% relative to the weight of the composition, and the amount of one or more binders is from 4 to 12% relative to the weight of the composition, and the amount of one or more disintegrants is from 2 to 6% relative to the weight of the composition, and the amount of one or more antiadherents and lubricants from 1 to 2% relative to the weight of the composition.
